(19) United States
(12) Patent Application Publication
(10) Pub. No.: US 2023/0087397 A1
(43) Pub. Date: Mar. 23, 2023

(54) PERSONNEL TRACKING AND DISINFECTING SYSTEM AND METHOD

(71) Applicant: DELTA ELECTRONICS, INC., Taoyuan City (TW)

(72) Inventors: Chia-Jen CHENG, Taoyuan City (TW); Chih-Wei TSAI, Taoyuan City (TW); Chih-Hsien WANG, Taoyuan City (TW)

(21) Appl. No.: 17/646,141

(22) Filed: Dec. 27, 2021

(30) Foreign Application Priority Data
Sep. 10, 2021 (CN) 202111060176.2

(51) Int. Cl. A61L 2/00; A61L 2/24; G06F 21/32

(52) U.S. Cl. CPC A61L 2/0005; A61L 2/24; G06F 21/32; A61L 2202/14; A61L 2202/25

(57) ABSTRACT

A personnel tracking and disinfecting system is applied to a place and includes recognition devices, at least one disinfecting device, storage and central controller. The central controller is configured to: control the recognition devices to recognize the person in the place; determine the position of the person in the place by cooperating with the recognition devices; activate the disinfecting device in the main area to disinfect the person in the main area, generate a disinfecting record based on recognition result generated by the recognition device in the main area, and store the disinfecting record in the storage; and determine whether the person in the extended area has the disinfecting record based on recognition result generated by the recognition device in the extended area.

### CROSS-REFERENCE TO RELATED APPLICATION

This application claims priority to China Application Serial Number 202111060176.2, filed Sep. 10, 2021, which is herein incorporated by reference in its entirety.

### BACKGROUND

### Field of Invention

This disclosure relates to personnel tracking and disinfecting system and method, and in particular to personnel tracking and disinfecting system and method applied to a place.

### Description of Related Art

For reducing the infection risk of epidemic disease, current approaches mostly implement the control of disinfection and name registration at main entrance/exit of building. However, current approaches are prone to problems. For example, the name registration reduces efficiency of person passing entrance/exit, person may enter place without disinfection and cannot be disinfected, and the disinfection is limited to spraying hands with alcohol.

### SUMMARY

An aspect of present disclosure relates to a personnel tracking and disinfecting system. The personnel tracking and disinfecting system is applied to a place and includes a plurality of recognition devices, at least one disinfecting device, storage and a central controller. The place includes a main area and at least one extended area. The recognition devices are configured to recognize person in the place to obtain person identity correspondingly. The disinfecting device is configured to disinfect the person in the place, wherein the main area is provided with a first recognition device and the disinfecting device, and the extended area is provided with a second recognition device. The storage is configured to store a personnel disinfecting record database. The central controller is coupled to the recognition devices, the disinfecting device and the storage and is configured to: control the recognition devices to recognize the person in the place; determine the position of the person in the place by cooperating with the recognition devices; activate the disinfecting device to disinfect the person in the main area, generate a disinfecting record based on a recognition result generated by the first recognition device, and store the disinfecting record in the personnel disinfecting record database; and determine whether the person in the extended area has the disinfecting record based on a recognition result generated by the second recognition device.

Another aspect of present disclosure relates to a personnel tracking and disinfecting method. The personnel tracking and disinfecting method is applied to a personnel tracking and disinfecting system, wherein the personnel tracking and disinfecting system is arranged in a place and includes a plurality of recognition devices, at least one disinfecting device, a storage and a central controller, the place includes a main area and at least one extended area, the main area is provided with a first recognition device and the disinfecting device, and the extended area is provided with a second recognition device. The method includes: by the central controller, controlling the recognition devices to recognize person in the place to obtain person identity correspondingly; by the central controller, determining the position of the person in the place by cooperating with the recognition devices; by the central controller, activating the disinfecting device to disinfect the person in the main area, generating a disinfecting record based on a recognition result generated by the first recognition device, and storing the disinfecting record in a personnel disinfecting record database; and by the central controller, determining whether the person in the extended area has the disinfecting record based on a recognition result generated by the second recognition device.

### BRIEF DESCRIPTION OF THE DRAWINGS

FIG. 1 is a block diagram of a personnel tracking and disinfecting system in accordance with some embodiments of the present disclosure;

FIG. 2 is a schematic diagram of the personnel tracking and disinfecting system when being arranged in a place in accordance with some embodiments of the present disclosure; and

FIG. 3 is a flow diagram of a personnel tracking and disinfecting method in accordance with some embodiments of the present disclosure.

### DETAILED DESCRIPTION

The embodiments are described in detail below with reference to the appended drawings to better understand the aspects of the present disclosure. However, the provided embodiments are not intended to limit the scope of the disclosure, and the description of the structural operation is not intended to limit the order in which they are performed. Any device that has been recombined by components and produces an equivalent function is within the scope covered by the disclosure.

The terms used in the entire specification and the scope of the patent application, unless otherwise specified, generally have the ordinary meaning of each term used in the field, the content disclosed herein, and the particular content.

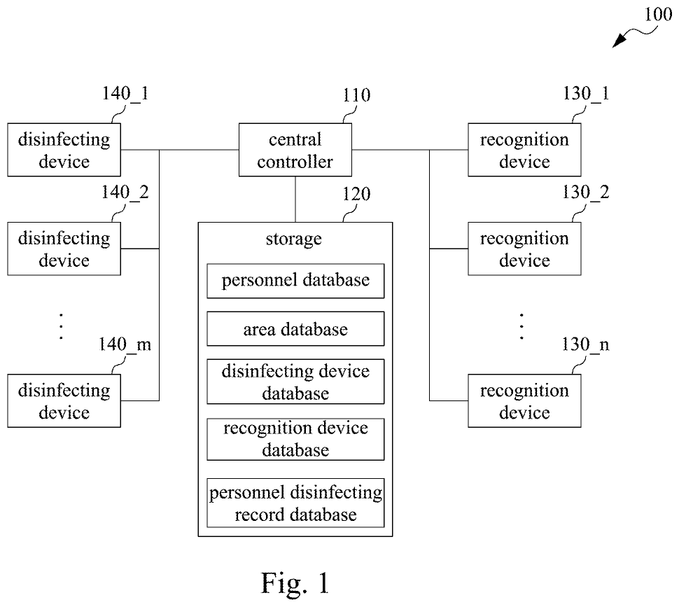

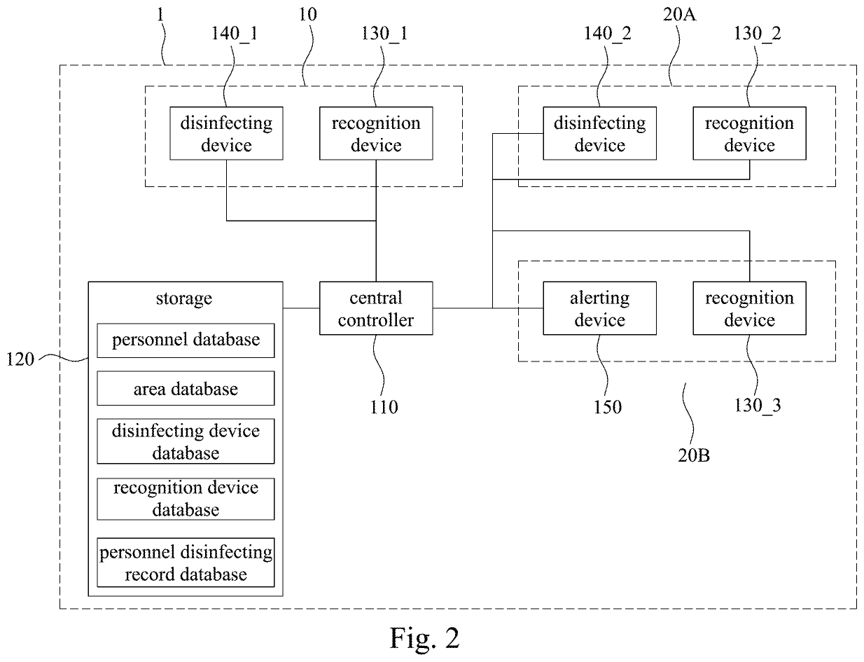

Referring to FIGS. 1-2, FIG. 1 is a block diagram of a personnel tracking and disinfecting system 100 in accordance with some embodiments of the present disclosure. FIG. 2 is a schematic diagram of the personnel tracking and disinfecting system 100 when being arranged in a place 1. When the personnel tracking and disinfecting system 100 is arranged in the place 1, the personnel tracking and disinfecting system 100 can be configured to monitor and manage the disinfecting state of personnel entering the place 1.

In some embodiments, as shown in FIG. 1, the personnel tracking and disinfecting system 100 includes a central controller 110, storage 120, a plurality of recognition devices 130_1-130_n and a plurality of disinfecting devices 140_1-140_m. The central controller 110 is coupled to the storage 120, the recognition devices 130_1-130_n and the disinfecting devices 140_1-140_m and is configured to control the storage 120, the recognition devices 130_1-130_n and the disinfecting devices 140_1-140_m. In other embodiments, the personnel tracking and disinfecting system 100 further includes at least one alerting device 150 (as shown in FIG. 2), and the alerting device 150 is also coupled to the central controller 110.

It can be appreciated that “m” or “n” as used herein represent the number of the disinfecting devices and the number of the recognition devices respectively. Both “m” and “n” are positive integer greater than 0, and can be same or different from each other.

In some embodiments, the central controller 110 can be implemented by one or more central processing unit (CPU), application-specific integrated circuit (ASIC), microprocessor, system on a Chip (SoC) or other suitable processing units.

In some embodiments, the storage 120 is configured to store a personnel database, an area database, a disinfecting device database, a recognition device database and a personnel disinfecting record database. The personnel database records the person identity (e.g., name, employee number, etc.) and/or the identification information (e.g., face image, serial number of RFID (radio frequency identification) tag, fingerprint image, etc.) of personnel which are recognizable by the recognition devices 130_1-130_n. The area database records all areas in the place 1 and the geographical relevance of the adjacent area of each area. The disinfecting device database records the identification information (e.g., device number) and the position of the disinfecting devices 140_1-140_m. The recognition device database records the identification information (e.g., device number) and the position of the recognition devices 130_1-130_n. The personnel disinfecting record database records the disinfecting record of the person in the place 1, and the disinfecting record each includes information such as person identity, disinfecting time and/or disinfecting location. It can be appreciated that if a person in the place 1 does not have the corresponding disinfecting record in the personnel disinfecting record database, it means that the person is not disinfected. In some embodiments, the storage 120 can be implemented by server.

In some embodiments, the recognition devices 130_1-130_n each is configured to recognize person in the place 1 to generate a recognition result. The recognition result can be face image, serial number of RFID tag and/or fingerprint image. Accordingly, the central controller 110 can compare the recognition result with a number of identification information in the personnel database, to obtain the person identity of the recognized person. In some embodiments, the recognition devices 130_1-130_n can be implemented by face recognition camera, short distance RFID reader and/or fingerprint identifier.

In some embodiments, the disinfecting devices 140_1-140_m each is configured to disinfect person in the place 1. In other embodiments, the disinfecting devices 140_1-140_m can be configured to disinfect the areas in the place 1. When one of the disinfecting devices 140_1-140_m is activated to disinfect, the central controller 110 can search the disinfecting device database according to the identification information of the one of the disinfecting devices 140_1-140_m, to obtain the disinfecting time and the disinfecting location correspondingly. In some embodiments, the disinfecting devices 140_1-140_m can be implemented by 222 nm of ultraviolet light irradiation device and/or alcohol spray device.

As shown in FIG. 2, the place 1 includes a main area 10 and at least one extended area (e.g., extended area 20A, extended area 20B) different from the main area 10. The main area 10 and the extended area are connected to each other. In some practical applications, the place 1 is office building, the main area 10 is reception lobby at ground floor or other main entrance/exit, and the extended area is conference room, tea room or working area. It can be appreciated that two extended areas 20A and 20B as shown in FIG. 2 are only for convenience and simplification of description and the number of the extended areas is not limited to those of FIG. 2.

Being the main entrance/exit of the place 1, the main area 10 is usually provided with one of the disinfecting devices 140_1-140_m and one of the recognition devices 130_1-130_n. In comparison with the main area 10, the extended area may not be provided with one of the disinfecting devices 140_1-140_m. In the embodiment of FIG. 2, the main area 10 is provided with the disinfecting device 140_1 and the recognition device 130_1, the extended area 20A is provided with the disinfecting device 140_2 and the recognition device 130_2, and the extended area 20B is provided with the alerting device 150 and the recognition device 130_3.

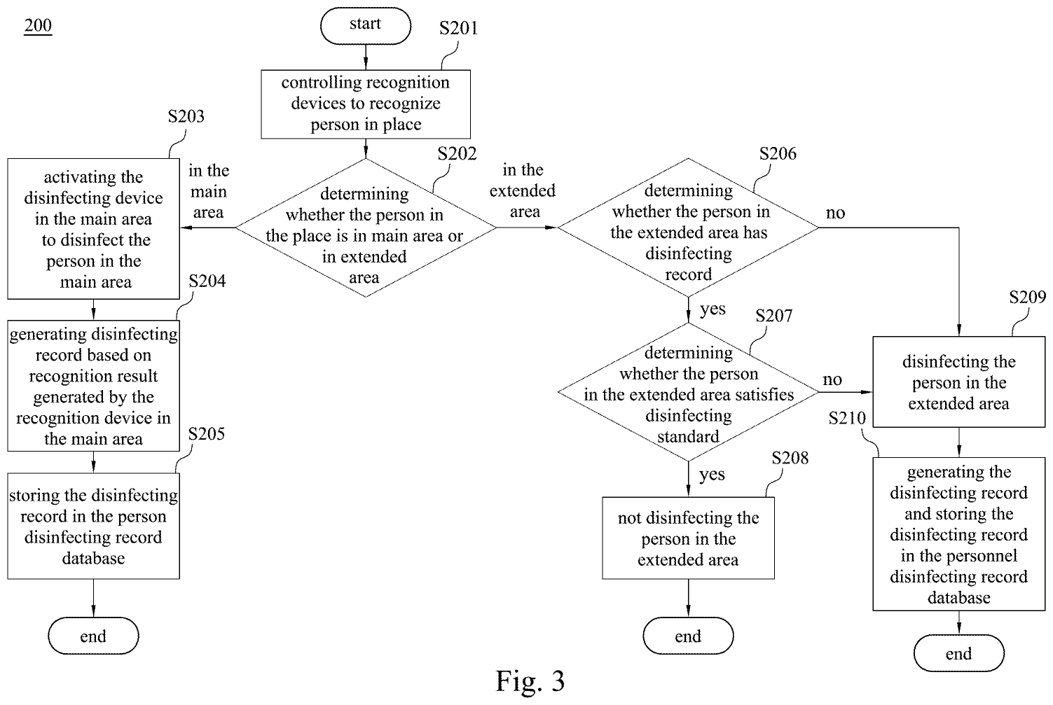

Referring to FIG. 3, FIG. 3 is a flow diagram of a personnel tracking and disinfecting method 200 in accordance with some embodiments of the present disclosure. The personnel tracking and disinfecting method 200 can be executed by the central controller 110 of FIG. 1 or 2. However, the present disclosure is not limited herein. For better understanding the present application, the personnel tracking and disinfecting method 200 would be described below with reference to FIGS. 2-3. As shown in FIG. 3, the personnel tracking and disinfecting method 200 includes operations S201-S210.

In operation S201, the central controller 110 controls the recognition devices 130_1-130_n to recognize the person in the place 1. In operation S202, the central controller 110 determines whether the person in the place 1 is in the main area 10 or in the extended area (i.e., the central controller 110 determines the position of the person in the place 1 by cooperating with the recognition devices 130_1-130_n).

In some embodiments, if the person is in the main area 10, the recognition device 130_1 in the main area 10 can recognize the person and transmit the recognition result to the central controller 110. Accordingly, the central controller 110 can search the recognition device database according to the identification information of the recognition device 130_1, so as to obtain that the recognized person is in the main area 10. In other embodiments, if the person is in the extended area 20A or 20B, the recognition device 130_2 or 130_3 in the extended area 20A or 20B can recognize the person and transmit the recognition result to the central controller 110. Accordingly, the central controller 110 can search the recognition device database according to the identification information of the recognition device 130_2 or 130_3, so as to obtain that the recognized person is in the extended area 20A or 20B. In brief, the central controller 110 can search the recognition device database according to the identification information of the recognition device which transmits the recognition result, so as to obtain the position of the recognized person.

In operation S203, if determining that the person is in the main area 10, the central controller activates the disinfecting device 140_1 in the main area 10 to disinfect the person in the main area 10. In operation S204, the central controller 110 generates the disinfecting record based on the recognition result generated by the recognition device 130_1 in the main area 10. In some embodiments, the central controller 110 obtains the person identity of the recognized person according to the recognition result generated by the recognition device 130_1 and further obtains the disinfecting time and the disinfecting location of the recognized person according to the disinfecting device 140_1 operating in the main area 10, so as to generate the disinfecting record.

In operation S205, the central controller 110 stores the disinfecting record in the personnel disinfecting record database. In some embodiments, the central controller 110 searches the personnel disinfecting record database according to the person identity in the disinfecting record generated currently, so as to determines whether a previous disinfecting record of the recognized person is stored in the personnel disinfecting record database. If the previous disinfecting record of the recognized person exists, the central controller 110 updates the disinfecting time and the disinfecting location in the previous disinfecting record according to the disinfecting record generated currently. If the previous disinfecting record of the recognized person does not exist, the central controller 110 adds the disinfecting record generated currently to the personnel disinfecting record database.

In operation S206, if determining that the person is in the extended area, the central controller 110 determines whether the person in the extended area has the disinfecting record. In some embodiments, the recognized person is in the extended area 20A as shown in FIG. 2. Accordingly, the central controller 110 obtains the person identity of the recognized person according to the recognition result generated by the recognition device 130_2 and searches the personnel disinfecting record database according to the person identity of the recognized person, so as to determine whether a previous disinfecting record of the recognized person is stored in the personnel disinfecting record database.

In operation S207, if determining that the person in the extended area 20A has the disinfecting record, the central controller 110 further determines whether the person in the extended area 20A satisfies a disinfecting standard. In some embodiments, the central controller 110 calculates an elapsed time according to a previous disinfecting time in the previous disinfecting record stored in the personnel disinfecting record database. For example, the central controller 110 subtracts the previous disinfecting time from a current time to calculate the elapsed time. After calculating the elapsed time, the central controller 110 compares the elapsed time with a first predetermined time (e.g., 1 hour).

If the elapsed time does not exceed the first predetermined time (i.e., satisfies the disinfecting standard), the central controller 110 executes operation S208. In operation S208, the central controller 110 does not disinfect the person in the extended area 20A. In other words, the central controller 110 does not activate the disinfecting device 140_2 in the extended area 20A. If the elapsed time exceeds the first predetermined time (i.e., does not satisfy the disinfecting standard), the central controller 110 labels the person in the extended area 20A as needing disinfection (for example, adds information and/or label related to “needing disinfection” to the disinfecting record of the person in the extended area 20A) and executes operation S209 simultaneously. In operation S209, the central controller 110 disinfects the person in the extended area 20A by activating the disinfecting device 140_2 in the extended area 20A.

In other embodiments, the previous disinfecting record of the recognized person further includes an execution time. In some practical applications, the recognized person was irradiated by the ultraviolet light irradiation device (i.e., one of the disinfecting devices 140_1-140_m) for 3 seconds before entering the extended area 20A, and therefore the execution time recorded in the disinfecting record by the central controller 110 is 3 seconds. Accordingly, in operation S207, the central controller 110 determines whether the execution time in the previous disinfecting record exceeds a second predetermined time (e.g., 5 seconds), so as to determine whether the person in the extended area 20A satisfies the disinfecting standard.

If the executed time exceeds the second predetermined time (i.e., satisfies the disinfecting standard), the central controller 110 executes operation S208. If the executed time does not exceed the second predetermined time (i.e., does not satisfy the disinfecting standard), the central controller 110 labels the person in the extended area 20A as needing disinfection and executes operation S209 simultaneously. The description of operations S208 and S209 are similar to those of above embodiments, and therefore are omitted herein.

In operation S210, the central controller 110 generates the disinfecting record and stores the disinfecting record in the personnel disinfecting record database. For example, the central controller 110 obtains the disinfecting time and the disinfecting location of the recognized person according to the disinfecting device 140_2 operating in the extended area 20A and updates the disinfecting time and the disinfecting location in the previous disinfecting record according to the person identity obtained in operation S206.

In another embodiment, the central controller 110 determines that the person in the extended area 20A does not have the disinfecting record in operation S206, so as to control the disinfecting device 140_2 in the extended area 20A to disinfect the person in the extended area 20A (i.e., operation S209). In operation S210, the central controller 110 obtains the disinfecting time and the disinfecting location of the recognized person according to the disinfecting device 140_2 operating in the extended area 20A and generates the disinfecting record based on the person identity obtained in operation S206, the disinfecting time and the disinfecting location. Then, the central controller 110 adds the disinfecting record generated currently to the personnel disinfecting record database.

In another embodiment, the central controller 110 determines the recognized person is in the extended area 20B as shown in FIG. 2 in operation S202, and determines that the person in the extended area 20B requires to be disinfected in operation S206 or S207. Accordingly, operation S209 is executed. As shown in FIG. 2, since the extended area 20B is not provided with the disinfecting device, the central controller 110 controls the alerting device 150 to inform the person in the extended area 20B to go to other nearby extended area (for example but not limited to the extended area 20A) which is provided with the disinfecting device, so as to disinfect the person by the disinfecting device in other extended area. Then, the central controller 110 generates the disinfecting record and stores the disinfecting record in the personnel disinfecting record database (i.e., operation S210). The description of operation S210 is similar to those of above embodiments, and therefore is omitted herein. In some embodiments, the alerting device 150 can be implemented by speaker, display or other suitable output devices, so that the central controller 110 may inform person in the place 1 by playing audio and/or video.

In yet another embodiment, the person in the place 1 further passes through at least one other area (not shown) before entering the extended area 20A or 20B (or other extended area), and the central controller 110 determines that the person in the extended area 20A or 20B requires to be disinfected. Accordingly, the central controller 110 can further control the disinfecting device arranged in the at least one other area to disinfect the at least one other area. In other words, the central controller 110 can disinfect the path along which the person moves before entering the extended area, so as to further reduce the disease infection risk.

In sum, by the recognition devices 130_1-130_n and the disinfecting device 140_1-140_m arranged pervasively in the place 1, the personnel tracking and disinfecting system 100 and the personnel tracking and disinfecting method 200 of the present disclosure disinfect person in the place 1 and generate the disinfecting record of the person. In addition, the personnel tracking and disinfecting system 100 can further determine whether the person in the place 1 is disinfected or satisfies the predetermined disinfecting standard according to the disinfecting record stored in the database, so as to activate the disinfecting device selectively.

Although the present disclosure has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present disclosure without departing from the scope or spirit of the invention. In view of the foregoing, it is intended that the present invention cover modifications and variations of this invention provided they fall within the scope of the following claims.

What is claimed is:

1. A personnel tracking and disinfecting system, applied to a place, wherein the place comprises a main area and at least one extended area, and the system comprises:
a plurality of recognition devices configured to recognize person in the place to obtain person identity correspondingly;
at least one disinfecting device configured to disinfect the person in the place, wherein the main area is provided with a first recognition device and the disinfecting device, and the extended area is provided with a second recognition device;
a storage configured to store a personnel disinfecting record database; and
a central controller coupled to the recognition devices, the disinfecting device and the storage and configured to:
control the recognition devices to recognize the person in the place;
determine the position of the person in the place by cooperating with the recognition devices;
activate the disinfecting device to disinfect the person in the main area, generate a disinfecting record based on a recognition result generated by the first recognition device, and store the disinfecting record in the personnel disinfecting record database; and
determine whether the person in the extended area has the disinfecting record based on a recognition result generated by the second recognition device.

2. The personnel tracking and disinfecting system of claim 1, wherein the central controller is further configured to:
determine whether the person in the extended area satisfies a disinfecting standard according to the disinfecting record; and
label the person which does not satisfy the disinfecting standard.

3. The personnel tracking and disinfecting system of claim 2, wherein the disinfecting standard is that an elapsed time after a previous disinfection does not exceed a first predetermined time.

4. The personnel tracking and disinfecting system of claim 2, wherein the disinfecting standard is that an execution time of a previous disinfection exceeds a second predetermined time.

5. The personnel tracking and disinfecting system of claim 2, wherein the extended area is further provided with an alerting device, the central controller is further configured to:
control the alerting device to inform; and
control the disinfecting device to disinfect the person.

6. The personnel tracking and disinfecting system of claim 2, wherein the person passes through at least one other area before entering the extended area, and the central controller is further configured to control the disinfecting device arranged in the at least one other area to disinfect.

7. A personnel tracking and disinfecting method, applied to a personnel tracking and disinfecting system, wherein the personnel tracking and disinfecting system is arranged in a place and comprises a plurality of recognition devices, at least one disinfecting device, a storage and a central controller, the place comprises a main area and at least one extended area, the main area is provided with a first recognition device and the disinfecting device, the extended area is provided with a second recognition device, and the method comprises:
by the central controller, controlling the recognition devices to recognize person in the place to obtain person identity correspondingly;
by the central controller, determining the position of the person in the place by cooperating with the recognition devices;
by the central controller, activating the disinfecting device to disinfect the person in the main area, generating a disinfecting record based on a recognition result generated by the first recognition device, and storing the disinfecting record in a personnel disinfecting record database; and
by the central controller, determining whether the person in the extended area has the disinfecting record based on a recognition result generated by the second recognition device.

8. The personnel tracking and disinfecting method of claim 7, wherein after operation of determining whether the person in the extended area has the disinfecting record, the method further comprises:
determining whether the person in the extended area satisfies a disinfecting standard according to the disinfecting record; and
labeling the person which does not satisfy the disinfecting standard.

9. The personnel tracking and disinfecting method of claim 8, wherein the disinfecting standard is that an elapsed time after a previous disinfection does not exceed a first predetermined time.

10. The personnel tracking and disinfecting method of claim 8, wherein the disinfecting standard is that an execution time of a previous disinfection exceeds a second predetermined time.

11. The personnel tracking and disinfecting method of claim 8, wherein the extended area is further provided with an alerting device, and the method further comprises:
by the central controller, controlling the alerting device to inform; and
by the central controller, controlling the disinfecting device to disinfect the person.

12. The personnel tracking and disinfecting method of claim 8, wherein the person passes through at least one other area before entering the extended area, and the method further comprises:
by the central controller, controlling the disinfecting device arranged in the at least one other area to disinfect.
